(19) United States
(12) Patent Application Publication
(10) Pub. No.: US 2016/0143514 A1
(43) Pub. Date: May 26, 2016

(54) SPHERICAL MECHANISM FOR MAGNETIC MANIPULATION

(71) Applicant: UNIVERSITY OF UTAH RESEARCH FOUNDATION, Salt Lake City, UT (US)

(72) Inventors: Arthur W. Mahoney, Salt Lake City, UT (US); Samuel W. Wright, Salt Lake City, UT (US); Jacob J. Abbott, Salt Lake City, UT (US)

(21) Appl. No.: 14/898,074

(22) PCT Filed: Jun. 12, 2014
(86) PCT No.: PCT/US2014/042143

§ 371 Date: Dec. 11, 2015

Related U.S. Application Data
(60) Provisional application No. 61/834,387, filed on Jun. 12, 2013.

(51) Int. Cl. A61B 1/00; A61B 34/30; A61B 5/06; A61B 1/04

(52) U.S. Cl. CPC A61B 1/00158; A61B 1/041; A61B 34/30; A61B 5/062

(57) ABSTRACT

A magnetic manipulation device (16) can include a housing (20), a spherical magnetic body (22) contained within the housing (20), and a plurality of sensors to detect the direction of the magnetic dipole of the spherical magnetic body (22). The spherical magnetic body (22) can be rotatable about a sphere axis of rotation which is (24a, 24b, 24c) can be in contact with the spherical magnetic body (22) to rotate the spherical magnetic body (22) about the sphere axis of rotation.

### RELATED APPLICATION

This application claims priority to U.S. Provisional Patent Application No. 61/834,387, filed on Jun. 12, 2013, which is incorporated herein by reference.

### GOVERNMENT INTEREST

This invention was made with government support under Grant #0952718 awarded by the National Science Foundation. The Government has certain rights in the invention.

### BACKGROUND

Untethered magnetic devices, such as magnetic microrobots and magnetically actuated capsule endoscopes, have become an active area of research because of the potential impact to minimally invasive medicine. These devices typically consist of some form of mechatronic or micro electromechanical systems (MEMS) device with a rigidly attached magnetic body on which magnetic forces and torques are applied by an external field. Some approaches to actuation utilize magnetic forces for pulling while others apply torque generated by rotating magnetic fields to roll on a surface, swim through a fluid, crawl through a lumen via helical propulsion, or screw through soft tissue. Because these devices can be viewed as simple end-effectors of a larger robotic system, and they may range in size from the microscale to the mesoscale, they are referred to herein as magnetically actuated tools (MATs) without any implied size.

The ability to control untethered MATs using a single rotating permanent magnet (RPM) has previously been shown where a rotating MAT can be propelled by a single RPM with the RPM placed in any position relative to the MAT, provided a specific position-dependent RPM rotation axis is established. In some experiments, an RPM can be rotated by a DC motor that is rigidly mounted to the tool-frame of an industrial six-degree-of-freedom (6-DOF) robotic manipulator. In this setup, the rotation axis of the RPM is fixed with respect to the tool-frame of the robotic manipulator. Such methods are capable of placing the RPM with a correct rotation axis to guide a MAT through relatively simple trajectories. However, when tasked with navigating a MAT through tortuous paths, the physical constraints of the robotic manipulator (i.e., joint limits and singularities) limit how the MAT can be actuated. To propel a MAT through a tortuous path, the RPM's rotation axis may undergo large continuous changes in direction, which may require the robotic manipulator to move into unfavorable configurations, known as singularities. Such singularities can result in temporary loss of control of the MAT position while the manipulator readjusts to a more favorable position.

### SUMMARY

The present technology provides a mechatronic device that rotates a spherical magnetic body to act as an RPM for the control of MATs. Accordingly, a magnetic manipulation device can comprise a housing and a spherical magnetic body contained within the housing. The spherical magnetic body can be rotatable about a sphere axis of rotation which is omnidirectionally variable. Rotators can be attached to the housing and are in contact with the spherical magnetic body to rotate the spherical magnetic body about the sphere axis of rotation.

The present technology can include a system for manipulation of a magnetic capsule endoscope which includes a magnetic manipulation device. The system can further include a robotic arm movable along at least two axes and supporting the magnetic manipulation device. A magnetic field sensor can be proximal to the spherical magnetic body to measure a magnetic dipole direction of the spherical magnetic body. A processor can be used to cause movements of the robotic arm and the plurality of rotators based on a location of the magnetic capsule endoscope and the magnetic dipole direction of the spherical magnetic body.

There has thus been outlined, rather broadly, the more important features of the invention so that the detailed description thereof that follows may be better understood, and so that the present contribution to the art may be better appreciated. Additional variations and aspects of the invention can be appreciated from the following detailed description.

### BRIEF DESCRIPTION OF THE DRAWINGS

FIG. 1 illustrates a system for manipulation of a MAT in accordance with an embodiment of the present invention.

FIG. 2 is a side perspective view of a spherical magnetic manipulation device usable with the system of FIG. 1 in accordance with an embodiment of the present invention.

FIGS. 3A and 3B illustrate a multiple omniwheel configuration of the device of FIG. 2.

FIG. 3C is a cutaway view of FIG. 3B of an omniwheel engaged against a magnetic spherical magnet.

FIG. 3D is a side view of a spoke of FIG. 3C in accordance with an embodiment of the present invention.

FIGS. 4A, 4B and 4C are schematic illustrations of spherical magnetic bodies in accordance with embodiments of the present invention.

FIGS. 5A and 5B illustrate two arrangements of sensors that can be used to measure a direction of the dipole moment of a spherical magnetic body in accordance with embodiments of the present invention.

FIGS. 6A and 6B illustrate a multiple omniwheel configuration in accordance with another embodiment of the present invention.

FIGS. 6C and 6D illustrate a multiple omniwheel configuration in accordance with yet another embodiment of the present invention.

FIG. 7 illustrates an alternative omniwheel configuration in accordance with an embodiment of the present invention.

FIG. 8 is a flow chart of a method in accordance with an embodiment of the present invention.

FIG. 9 is a flow chart of a method in accordance with an embodiment of the present invention.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### DEFINITIONS

In describing and claiming the present invention, the following terminology will be used.

The singular forms “a,” “an,” and “the” include plural referents unless the context clearly dictates otherwise. Thus, for example, reference to “a housing” includes reference to one or more of such features and reference to “orienting” refers to one or more such steps.

As used herein with respect to an identified property or circumstance, “substantially” refers to a degree of deviation that is sufficient so as to measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

The present technology provides a mechatronic device that rotates a spherical magnetic body to act as an RPM for the control of other MATs. The device can generally utilize three rotators or omniwheels to enable holonomic control of an orientation of the spherical magnetic body, such that an instantaneous axis of rotation of the spherical body can be set arbitrarily. In one exemplary application, the device can be mounted as an end-effector of a simplified robotic manipulator that controls a Cartesian position of the device, and thus the RPM, without requiring a singularity-prone robotic wrist. The dipole moment of the RPM (i.e., the vector pointing from the south to the north magnetic poles, whose magnitude is equal to the strength of the magnet) can be estimated on-line, utilizing three or more sensors (e.g., Hall effect) which can be mounted to the device or otherwise located proximal to the device. The sensed RPM dipole is used for closed-loop control of the RPM's dipole vector axis of rotation. These and other aspects of the invention are described in more detail through the following exemplary description.

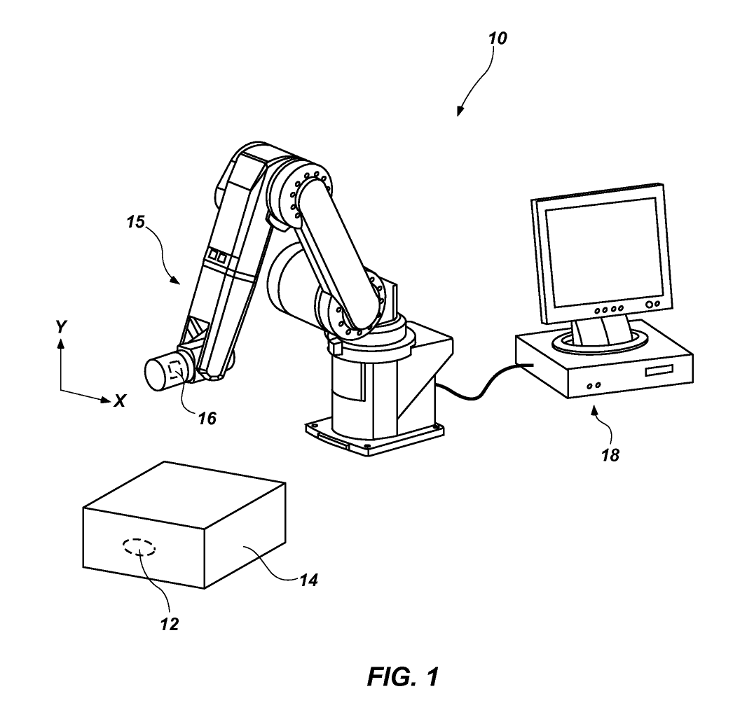

FIG. 1 shows a system 10 for manipulation of a magnetically actuated tool 12, such as a magnetic capsule endoscope, contained in a body 14 by utilizing a robotic arm 15 that is movable in at least two axes X and Y. A magnetic manipulation device 16 may be pivotally and rotatably mounted to and supported by a distal end of the robotic arm 15. A computer or other processor 18 is connected to the robotic arm 15 for control and manipulation of the robot and the magnetically actuated tool 12 via the magnetic manipulation device 16. A receiver (not shown) can be connected to the processor 18 and adapted to receive image and position data as they are emitted by the data transmitted by the magnetic capsule endoscope 12. In some embodiments, the magnetic capsule endoscope is introduced into the gastro-intestinal tract of a patient or other internal area of a patient. In other embodiments, the magnetically actuated tool may be introduced into other areas for additional applications, such as manipulating a magnetic surgical tool in the ventricular system of the human brain or in the lumen pathways of the cardiovascular, urinary, and pulmonary systems. It should be noted that the magnetic tool does not need to be untethered. For example, other applications can include manipulating a magnet-tipped catheter, guidewire, or other flexible tools for medical interventions. Other applications can also include industrial applications where MATs may be useful.

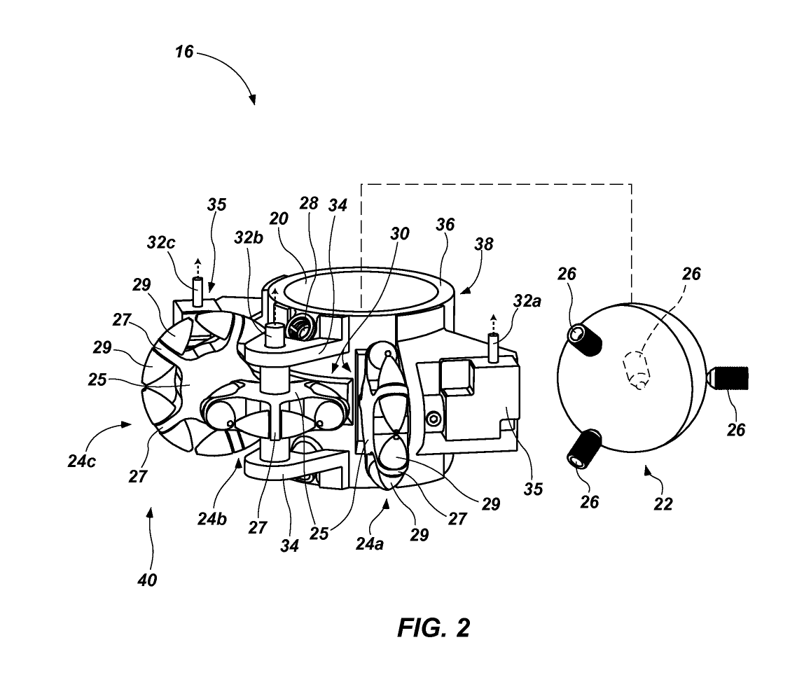

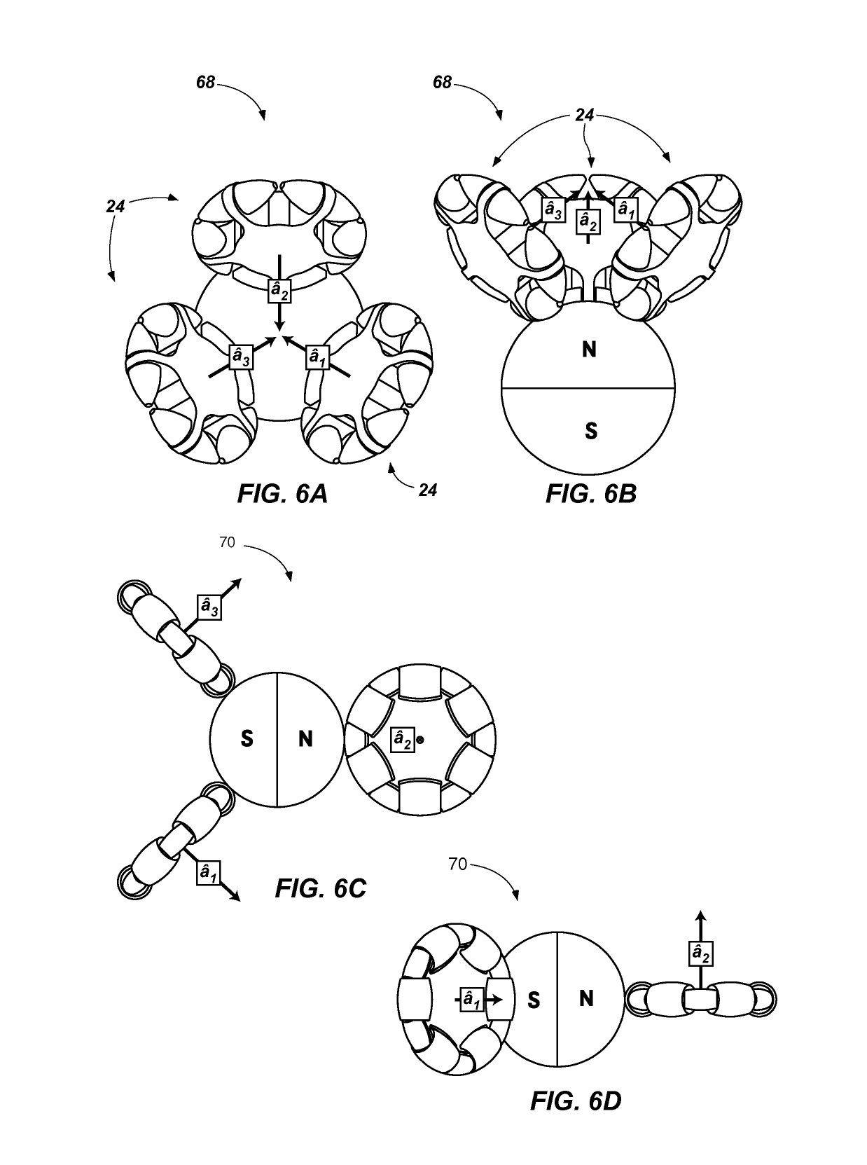

FIG. 2 shows an example magnetic manipulation device 16 that is usable with the system 10 of FIG. 1. The magnetic manipulation device 16 includes a cylindrical housing 20 and a spherical magnetic body 22 contained within the housing 20. The spherical magnetic body 22 is rotatable about a sphere axis of rotation which is omnidirectionally variable. A plurality of rotators 24a, 24b, 24c (collectively “24”) are adapted to rotate the spherical magnetic body 22 about the sphere axis of rotation. Further, the rotators can optionally be rotatably coupled to the housing 20, although the rotators can alternatively be attached to a separate support member which allows the rotators to rotate the spherical magnetic body 22. In the present example, rotators 24 are provided in the form of omniwheels. The axis of each omniwheel (â₁,â₂, and â₃) is orthogonal to a contact normal vector of the respective omniwheel and the spherical magnetic body 22, and all three omniwheel axes are mutually orthogonal, although other suitable configurations and rotators are possible (e.g., FIGS. 6A and 6B). Each omniwheel 24 includes a hub 25 with a plurality of spokes 27, such as five spokes made of aluminum. A pair of roller 29 is rotatably supported by each spoke 27 of each hub 25. Thus each omniwheel also includes rollers which each rotate on roller axes perpendicular to the respective omniwheel rotational axis. In this manner rotational force in a rotation direction of the omniwheel axis can be applied to the spherical magnetic body while allowing free rotation along directions transverse to the rotational direction.

In some aspects, physical constraints 26 are positioned around the spherical magnetic body 22, and rotatably contact the body 22, to prevent it from translation in space within the housing 20. The constraints 26 may be positioned through a plurality of spaced threaded apertures 28 of the housing 20. In some aspects, the constraints 26 are a set of four ball-roller tipped precision set screws. The tip of each constraint 26 may contain a freely rotating ball supported by many subrollers to reduce friction between the body 22 and the constraints 26. The constraints 26 can be adjustable via matching threaded apertures 28 (or inserts) in the body of the housing 20, and can be finely tuned in such a way as to allow spherical magnetic body 22 to freely rotate while allowing minimally perceptible translation.

The housing 20 includes openings 30 to permit the rotators 24 to at least partially extend through the housing 20 to rotatably contact the spherical magnetic body 22 within the housing. Controllable motors (not shown) can be mounted adjacent the magnetic manipulation device 16 (e.g., within a robotic end-effector) to drive each of the rotators 24. For example, the motors can be coupled to driveshafts 32a, 32b, and 32c. Driveshaft 32b extends through mounting brackets 34 of the housing 20 and drive omniwheel 24b. In the present example, each driveshaft 32a, 32b, and 32c is parallel to one another to eliminate the need of flexible driveshafts or other complicated forms of power transmission, although non-parallel driveshafts can be used. Driveshafts 32a and 32c can be connected to 90-degree gearboxes 35. Each gearbox 35 can optionally have a 1:1 gear ratio so as to match torque/velocity of the driveshaft 32b. The output of each of driveshaft can be optionally supported by integrated ball bearing pillow-block assemblies in order to reduce torque loss in the transmission.

As discussed with reference to FIG. 1, the magnetic manipulation device 16 may be mounted as a tool to a multi-DOF robotic manipulator, and optionally mounted at a surface 36 (shown in FIG. 2) of the device 16. In this manner, three orientation degrees-of-freedom can be added to an existing robotic arm. In the case of a 3-DOF Cartesian robot, for example, the magnetic manipulation device enables 6-DOF control of the magnetic dipole without any robot wrist. In this configuration the device 16 can be positioned so that a rear side 38 (FIG. 2) of the device 16 is oriented away from the workspace where a MAT 12 is under actuation. In this way, a front side 40 (which is streamlined of moving parts) is presented to the workspace, thereby reducing the risk of damage to the moving components and enabling the spherical magnetic body 22 to be positioned closer to a MAT 12.

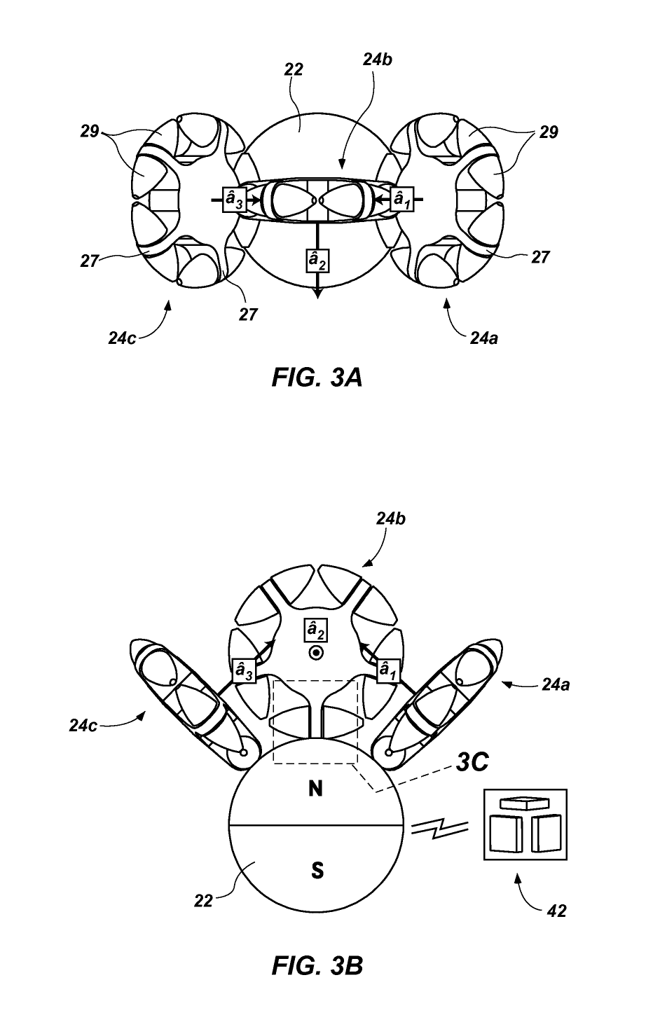

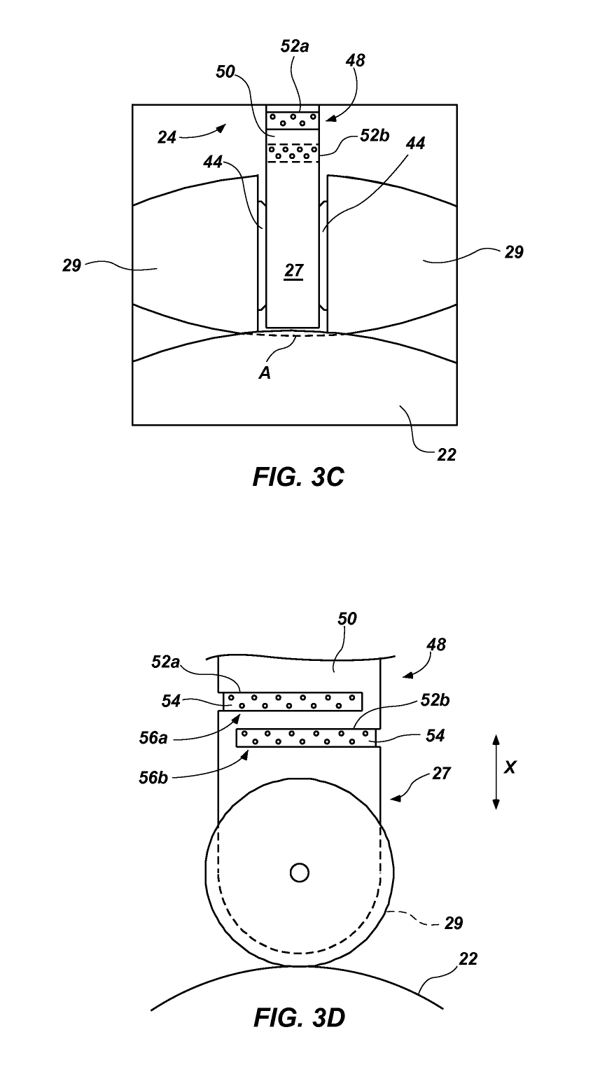

FIGS. 3A and 3B show the omniwheels 24 and magnetic spherical body 22 example configuration as discussed with reference to FIG. 2 but with the housing and supports removed for clarity. In this case, the axis of rotation for each omniwheel, â₁,â₂, and â₃, are oriented orthogonal to one another. However, non-parallel, non-orthogonal axes of rotation may be used by taking into account corresponding non-orthogonal force contributions.

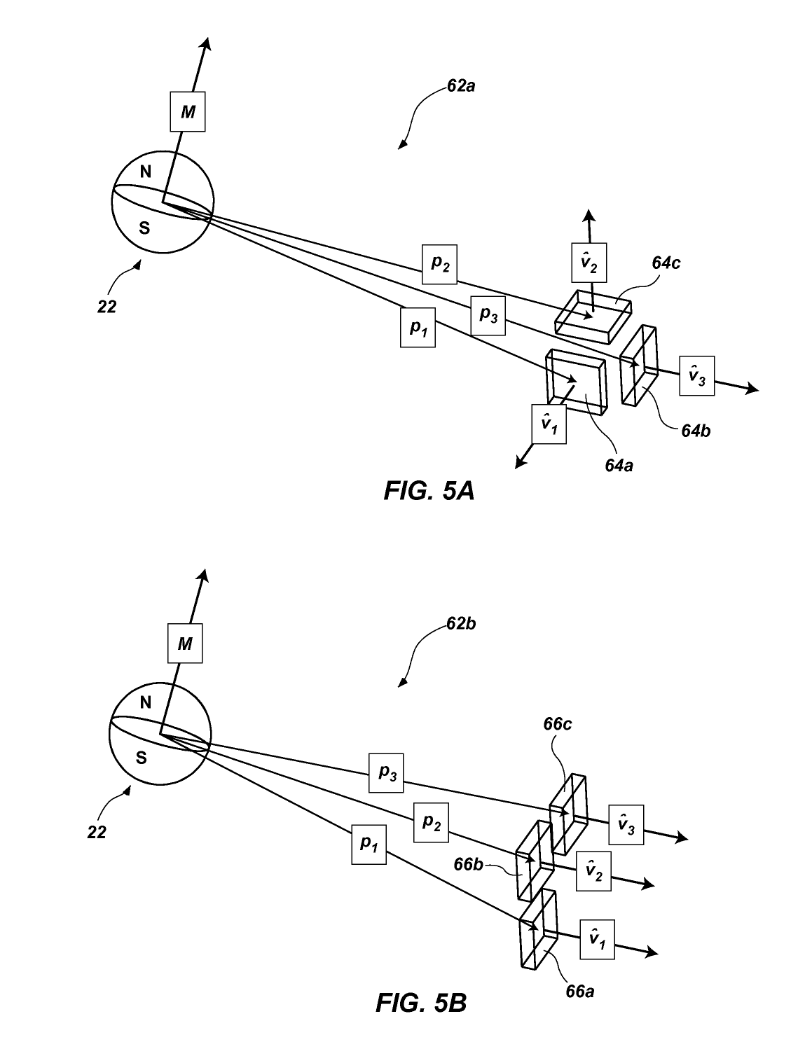

A magnetic field sensor 42 can be positioned proximate the magnetic spherical body 22 and measures the dipole moment of the body 22 for closed-loop control of the angular velocity and dipole orientation of the magnetic spherical body 22. Magnetic field sensor(s) can be coupled to the housing. In one example, two, three or more magnetic field sensors can be used to measure the dipole orientation of the spherical magnetic body. The sensor 42 can be used with an end-effector of a robotic manipulator performing remote magnetic-manipulation tasks, such as described with reference to FIG. 1. Possible sensor arrangements and operations will be discussed further below with reference to FIGS. 5A and 5B.

The magnetic manipulation device 16 (FIG. 2) enables holonomic singularity-free control of the orientation of the spherical magnetic body 22. As noted above, the spherical magnetic body 22 can be driven by way of three omniwheels 24 that contact the spherical magnetic body 22, whose collective axes form a full-rank basis for [symbol] ³. Because an omniwheel is a mechanism that enables free rotation about two axes and controlled rotation about a third axis, designing the three omniwheel axes to be linearly independent enables any instantaneous sphere rotation axis to be achieved. By making the axis of rotation of the spherical magnetic body 22 continuously variable (irrespective of the robotic manipulator that positions the device), the workspace constraints of a robotic manipulator are avoided, which also avoids undesirable singularities. This allows the robotic manipulator free to position itself for robust control of MAT locomotion, while leaving the axis of rotation of the RPM unrestricted and in the control of the device. This also enables simpler robotic manipulators to be considered (e.g., 3-DOF (degree of freedom) or 4-DOF gantry and SCARA (Selective Compliance Assembly Robot Arm) robots) with the same level of MAT manipulatability.

Other spherical manipulation devices have been previously demonstrated in “ballbot” systems in which a robot balances itself atop a sphere (e.g., a soccer ball) to achieve holonomic control, and inverted-pendulum based controllers are used to stabilize the robot. With ballbots, only the instantaneous angular velocity of the sphere is important for control (i.e., there is no preferred “north pole” of a soccer ball), so only open-loop control is required. However, for the control of a magnetic spherical body, knowledge of the magnet's dipole orientation is important, both for the establishment of the sphere's angular velocity (in which the dipole rotates such that it is perpendicular to the axis of rotation), as well as the orientation-control of the dipole itself for other non-rotating tasks.

Because knowledge of the dipole's orientation is important for the magnetic manipulation device 16 to function, the system includes the magnetic field sensor 42, which in some aspects is comprised of three or more Hall-effect sensors to estimate the dipole orientation of the rotating magnetic sphere (see e.g., FIGS. 5A and 5B). As introduced above, the magnetic spherical body 22 is contacted by three omniwheels 24 which actuate the rotation of the permanent spherical magnetic body 22, making the spherical body rotate according to input provided by a feedback unit (not shown) based on magnetic dipole direction and desired motion. Each omniwheel 24 contacts the spherical magnetic body 22 with the plurality of rollers 29 that allow the omniwheel 24 to roll with full force in the selected driving direction while allowing the spherical magnetic body 22 to roll perpendicular to the drive direction with minimal or negligible friction. Thus, the rollers 29 can typically freely spin. The speed at which each omniwheel 24 rotates is determined by the desired angular velocity Ω of the magnet, which is set by an external control system or user input. While the spherical magnetic body 22 rotates, the sensor 42 (FIG. 3B) that measures the magnetic field is used to determine the dipole moment vector (the vector from the south to north magnetic poles) of the spherical magnetic body 22. The measured dipole moment vector can be used for closed-loop control of the dipole orientation of the spherical magnetic body 22. The entire system (including the spherical magnetic body, the omniwheels, the actuation system for the omniwheels, the sensor system, and the support structure) can be presented in a compact package to be used as the end-effector of a robotic manipulator for controlling a MAT (e.g., a magnetic capsule endoscope).

For some desired angular velocity Ω of the spherical magnetic body 22, the omniwheel rotation speeds are determined. As an illustration of this approach, let the unit-length vectors \hat{d}ₒ₁, \hat{d}ₒ₂, and \hat{d}ₒ₃ point from the spherical magnetic body 22 center to the contact point where each of the three omniwheels 24 touches the spherical magnetic body 22. The omniwheel axes âₒ₁, âₒ₂, and âₒ₃ are assumed perpendicular to \hat{d}ₒ₁, \hat{d}ₒ₂, and \hat{d}ₒ₃, respectively, and that there is no slip between each omniwheel 24 and the spherical magnetic body 22. Given a magnet angular velocity Ω, the surface velocity of the magnet at the i^{th} omniwheel-magnet contact point is

uₒᵢ=rₛΩ×\hat{d}ₒᵢ,

where rₛ is the radius of the spherical magnetic body 22.

The components of uₒ₁, uₒ₂, and uₒ₃ parallel to each omniwheel axis are transferred directly into rotation of the omniwheel rollers 29, and cause no rotation of the omniwheels themselves. All other components of uₒ₁, uₒ₂, and uₒ₃ cause each omniwheel 24 to rotate with scalar rotation speeds ωₒ₁, ωₒ₂, and ωₒ₃, respectively. The component direction of uₒᵢ that cause causes the i^{th} omniwheel to rotate is

\hat{q}ₒᵢ=\hat{d}ₒᵢ×âₒᵢ.

Under the assumption of no-slip, the projection of uₒ₁, uₒ₂, and uₒ₃, onto the directions \hat{q}ₒ₁, \hat{q}ₒ₂, and \hat{q}ₒ₃, respectively, are mapped to the scalar rotation speeds of each omniwheel 24 by the reciprocal of each omniwheel's radius (denoted by rₒᵢ) as

$${ω}_{o 1} = \frac{1}{{r}_{o i}} {\hat{q}}_{oi}^{T} {u}_{oi} = \frac{{r}_{s}}{{r}_{oi}} {a}_{oi}^{T} {( {\hat{d}}_{oi} )}^{2} Ω$$

where [symbol] (d) is the skew-symmetric matrix defined as

$$( d ) = [ \begin{matrix}0 & - {d}_{z} & {d}_{y} \\ {d}_{z} & 0 & - {d}_{x} \\ - {d}_{y} & {d}_{x} & 0\end{matrix} ] . \\ with$$

$$d = {[ {d}_{x} {d}_{y} {d}_{z} ]}^{T}$$

All three omniwheel scalar rotation speeds can be packed into the vector ω and related to the spherical magnet angular velocity Ω, in matrix form as

$$ω = [ \begin{matrix}{ω}_{o 1} \\ {ω}_{o 2} \\ {ω}_{o 3}\end{matrix} ] = [ \begin{matrix}\frac{{r}_{s}}{{r}_{o 1}} {\hat{a}}_{o 1}^{T} \\ \frac{{r}_{s}}{{r}_{o 2}} {\hat{a}}_{o 2}^{T} \\ \frac{{r}_{s}}{{r}_{o 3}} {\hat{a}}_{o 3}^{T} \end{matrix} ] Ω = Ω .$$

The omniwheel axes and positioning are designed such that [symbol] has full rank, otherwise there will exist a direction of Ω that cannot be achieved with any selection of omniwheel rotation speeds. Although linear independence of the rows of [symbol] is a sufficient condition mathematically, in practice the rows can be designed to be as close as possible to being mutually orthogonal. Otherwise, some desired Ω will result in an unnecessarily, and possibly unachievably, large omniwheel rotation speed. FIGS. 3A, 3B, 6A, and 6B show two possible arrangements of omniwheels.

It is possible to violate the assumption that each vector \hat{d}ₒᵢ from the spherical magnet center to the contact point of the i^{th} omniwheel is not perpendicular to the i^{th} omniwheel axis âₒᵢ. However, this would require a different style of omniwheel to ensure that contact is continuously maintained through each omniwheel revolution in both the omniwheel's passive and active degrees-of-freedom.

FIG. 3C is a cutaway view of FIG. 3B of the surface-to-surface engagement between an omniwheel 24 and the spherical magnetic body 22. A ball bearing 44 can be placed in each spoke 27 allowing the rollers 29 to spin as freely as possible in their non-drive direction. Traction complications are often encountered due to gaps between rollers and a spherical surface, which causes periodic loss of traction during rotation. Thus, in some aspects, the rollers 29 are covered in a tough compliant rubber coating that improves surface-to-surface traction, although other suitable coverings can be used. Because of the compliance of the rubber coated rollers 29, the rollers 29 are at least partially constantly compressed against the spherical magnetic body 22 during their respective rotational sweep across the spherical body, which is illustrated by dashed line A that shows an imaginary, overlapping contact region between the pairs of roller 29 and the body 22. Such configuration contributes to substantially constant (and even absolute constant) surface-to-surface contact between the omniwheels and spherical magnetic body, except a small gap as one spoke sweeps away from the spherical body surface and an adjacent spoke sweeps towards the surface.

Another feature that contributes to maximized or absolute surface-to-surface contact between the omniwheels 24 and the spherical magnetic body 22 is a compliance unit 48 (also shown on FIG. 3D). The compliance unit 48 includes a compressible support arm 50 (i.e., a portion of a spoke 27) that includes a first cutout 52a and a second cutout 52b. The cutouts 52a, 52b oppose each other to form a serpentine-like profile. A compliant material 54, such as a silicone-based material, optionally substantially fills the areas 56a, 56b defined by the cutouts 52a, 52b such that the support arm 50 is compressible along a support axis X extending a length of the support arm 50. Therefore, compliance is added to each spoke, effectively turning the spokes into a suspension system for each omniwheel. Such arrangement further introduces a damping effect and prevents the hub and spokes from yielding during operation. Alternate solutions include incorporating compliance elsewhere in the device. For example, such compliance can be introduced into the rollers, between the omniwheel hubs and the spherical body (e.g., built into the structure), or built into the rolling set-screws, or combinations thereof.

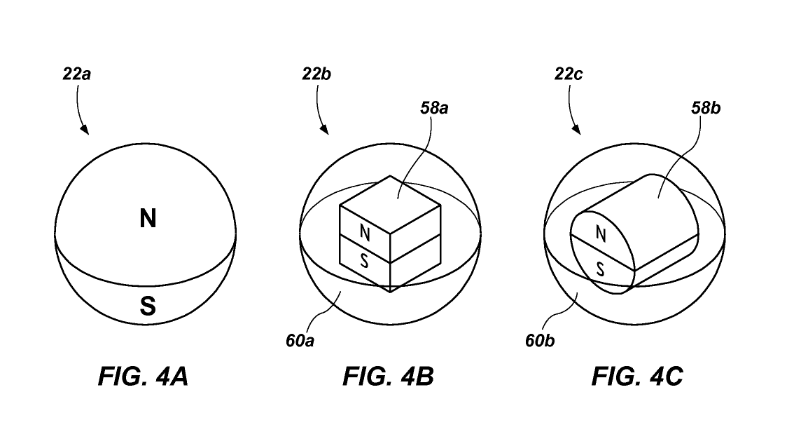

Although a solid homogeneous spherical magnetic body can be used, non-spherical magnetic portions can optionally be embedded or encapsulated in a spherical structure. FIGS. 4A, 4B and 4C show three spherical magnetic bodies 22a, 22b, 22c that can be used as the spherical magnetic body for any embodiment described in the present application. Body 22a is a spherical body with upper and lower hemispheres of north and south magnetic poles. In this case, the spherical magnetic body is a uniform composition magnetic body. Body 22b includes a cube-shaped magnet 58a encapsulated in a spherical shell 60a, and body 22c is a cylindrical-shaped magnet 58b encapsulated in a spherical shell 60a. Any suitable magnetic core shape can be used, as long as a sufficiently strong magnetic field can be generated, and the outer profile of the body is spherical. Alternatively, the spherical magnetic body can be coated with a durable coating (e.g. polymer, refractory metal, carbides, diamond-like carbon, etc.). Similarly, the outer surface of the spherical body can be optionally textured. For example, each of the spherical shells 60a, 60b can be made from two 3D-printed hemispheres with a textured surface (similar to frosted glass) to increase surface-to-surface traction between the omniwheels and the body, although other suitable spherical encapsulates could be used. The magnets 58a, 58b, and 22a are typically grade-N42 permanent magnets, but could be any suitable magnet or magnetic device.

Three or more sensors can be used to calculate dipole moment M of the spherical magnetic body. FIGS. 5A and 5B show schematics of two exemplary sensor arrangements 62a, 62b, respectively, that could be used to measure the direction of a magnetic body's dipole moment M. In both examples the sensor arrangements 62a, 62b are each comprised of three Hall effect sensors, such as the sensor 42 discussed with reference to FIG. 3B. In the configuration of FIG. 5A, all three sensors 64a, 64b, 64c are mutually orthogonal. If p₁, p₂, and p₃ are nearly the same (i.e., the sensors are approximately collocated), then the sensors effectively measure the magnetic field vector H at their common position and M can be found by inverting the point-dipole model, which is consistently invertible and well conditioned. Alternatively, the magnetic field sensors can be oriented in a same direction but are located at different positions or locations. For example, in the configuration of FIG. 5B, each sensor 66a, 66b, 66c faces the same direction. In this arrangement, the matrix S becomes rank deficient when the sensor positions converge.

The dipole moment of the magnetic body 22 (denoted by the vector M) is the vector from the south to north poles of the magnetic body 22. Methods of magnetic manipulation using a single permanent magnet typically require the magnet's dipole moment to be specifically directed and the moment to be known. The dipole moment M of the present the magnetic body 22 can be determined by measuring its magnetic field H.

Hall effect sensors measure the component of the field in the direction perpendicular to the sensor's face. The general case of n Hall-effect sensors can be assumed. In such case, each sensor of FIGS. 5A and 5B is positioned in space such that the vectors p₁ through pₙ measure each sensor's position relative to the spherical magnet's center, and \hat{v}₁, through \hat{v}ₙ are unit-magnitude vectors that describe the directions that are sensed by each sensor. All vectors are expressed in the same frame as M. The magnetic field at each sensor position is denoted by H₁ through Hₙ. The measured component of the field produced by the i^{th} sensor is denoted with the scalar sᵢ and is given by equation (4):

sᵢ=\hat{v}ᵢ^{T}Hᵢ

The magnetic field Hᵢ, at each sensor position pᵢ, can be predicted with the point-dipole model described by equation (5):

$${H}_{i} = \frac{1}{4 π {‖ {p}_{i} ‖}^{3}} ( 3 {\hat{p}}_{i} {\hat{p}}_{i}^{T} - I ) M = {H}_{i} M$$

where I is the 3×3 identity matrix. Equation (5) nearly exactly predicts the field produced by an ideal spherical permanent magnet, although imperfections in the magnet can cause minor variations. For all other geometries, it is an approximation that becomes more accurate with increasing distance. Substituting equation (5) into equation (4) produces an expression relating the magnet's dipole moment M to each of the n sensor measurements, which can be aggregated into the matrix equation (6):

$$S = [ \begin{matrix}{\hat{v}}_{1}^{T} {H}_{1} \\ ⋮ \\ {\hat{v}}_{n}^{T} {H}_{n}\end{matrix} ] M = S M$$

where S=[S₁ . . . sₙ]^{T}. The n×3 constant matrix S encapsulates the complete geometric description of the sensor arrangement, as it pertains to the estimation problem. If the matrix S has full column rank, then a solution for the dipole moment M can be found as equation (7):

M=S^{†}S

where S^{†}=VΣ^{†}U^{T} is the Moore-Penrose pseudo-inverse of S, using the singular-value decomposition S=UΣV^{T}, where the columns of U and V are the output and input singular vectors of S, respectively, Σ contains the singular values of S on the main diagonal and zeros elsewhere, and Σ^{†} is the transpose of Σ in which the positive singular values have been replaced by their reciprocals. The matrix S should be made to have full column rank by using at least three Hall-effect sensors and appropriately selecting the positions (pᵢ) and directions (\vec{v}ᵢ) of each sensor. When n>3, equation (7) provides the best estimate of M in a least-squares sense. The constant matrix S^{†} can be calculated off-line. When the sensors are rigidly attached to the device housing device and never move after the device has been built, then the matrix S can be determined and fixed based on the device onfiguration. However, when the sensors move in relation to the magnet center (e.g., Hall sensors are mounted to the robot arm) then the sensor matrix S may change over time. In this case, the matrix S can be constructed using equations 5 and 6 (or determined using a lookup table).

The vector of sensor measurements S can be modeled as a normal multivariate random process S˜N(μ, C) with mean vector μ and covariance matrix C. The sensor measurement distribution S is propagated through equation (7) to a normal multivariate random process of the measured dipole moment as in equation (8):

M˜N(S^{†}μ,S^{†}CS^{†T}).

Under the assumption that the sensor measurements are independent with the same variance ρ², the covariance matrix can be expressed as C=ρ² I, which simplifies the distribution of the measured dipole moment to equation (9):

M˜N(S^{†}μ,S^{†}CS^{†T}).

where A is the 3×3 diagonal submatrix of Σ with the singular values of S on its diagonal. Along with making S full column rank, the sensors can also be ideally arranged to minimize the variance of the measured dipole moment by decreasing the singular values of the dipole moment covariance (stored on the diagonal of Λ⁻¹), which is equivalent to maximizing the singular values of S.

FIGS. 6A and 6B illustrate an alternative multiple omniwheel configuration 68 in accordance with an embodiment of the present invention. It will be appreciated that the configuration 68 could be used with the various embodiments and configurations discussed in the present application. In particular, the housing arrangement discussed with reference to FIG. 2 can be used with the omniwheel configuration 68 such that the housing would have mounting brackets and driveshafts to drive each omniwheel 24. In the present example, the axis of each omniwheel 24 is orthogonal to the contact normal vector of the omniwheel 24 and the spherical magnet 22, and all three omniwheel axes (â₁,â₂, and â₃) are mutually orthogonal.

FIGS. 6C and 6D illustrate another multiple omniwheel configuration 70 with mutually orthogonal omniwheel axes (â₁,â₂, and â₃). In this configuration, the omniwheels have a center of rotation which lies along an equator of the spherical magnetic body 22. Specifically, two omniwheel axes lie within an equatorial plane, while the third omniwheel axes is perpendicular to the equatorial plane. Drive shafts and motors can be connected to each omniwheel. In one aspect, three motors can be oriented in a common direction such that each of two omniwheels are connected via an angular torque transfer shaft (e.g. 90°) in order to provide a more compact device profile.

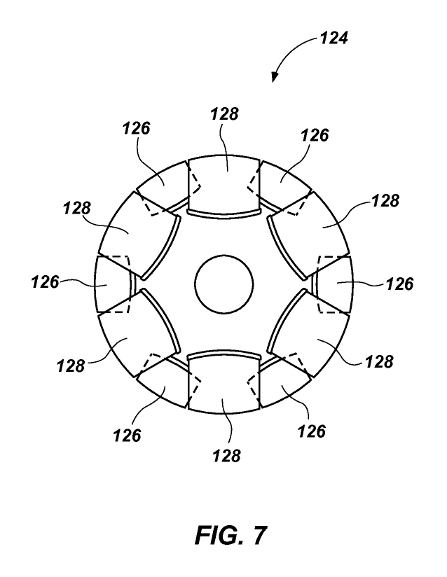

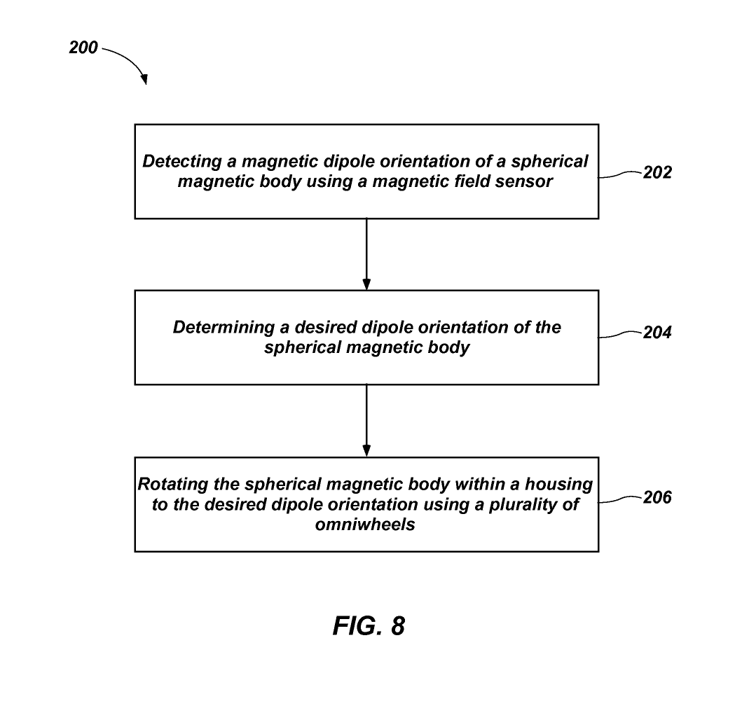

FIG. 7 shows a side plan view of an example omniwheel 124 having a plurality of nested rollers of differing diameter. For instance, small rollers 126 are alternately mounted between large rollers 128. Such configuration produces a nearly gap-free omniwheel that can be used to mitigate binding caused by large gaps which may occur with fewer spokes and fewer corresponding rollers. The omniwheel 124 could replace some or all of the omniwheels 24 discussed with the embodiments of FIGS. 2, 3, 5, 6A and 6B, similar to those shown in FIGS. 6C and 6D. As a general guideline, the omniwheels can include at least five spokes and rollers, and often from 5 to 20 spoke and roller assemblies, and most often from 5 to 15.

FIG. 8 shows a method 200 in accordance with an embodiment of the present invention. At step 202, the operation is performed of detecting a magnetic dipole orientation of a spherical magnetic body using a magnetic field sensor. At step 204, the operation is performed of determining a desired dipole orientation of the spherical magnetic body using a processor. Although other processing techniques can be used, one exemplary approach is described in U.S. patent application Ser. No. 14/223,510, filed Mar. 24, 2014, entitled “Manipulation of an Untethered Magnetic Device with a Magnet Actuator,” which is incorporated herein by reference. At step 206, the operation is performed of rotating the spherical magnetic body within a housing to the desired dipole orientation using a plurality of omniwheels. These steps can be performed utilizing all or some of the systems and components discussed in the present invention, with particular reference to FIGS. 1, 2, 5A, and 5B as exemplary embodiments.

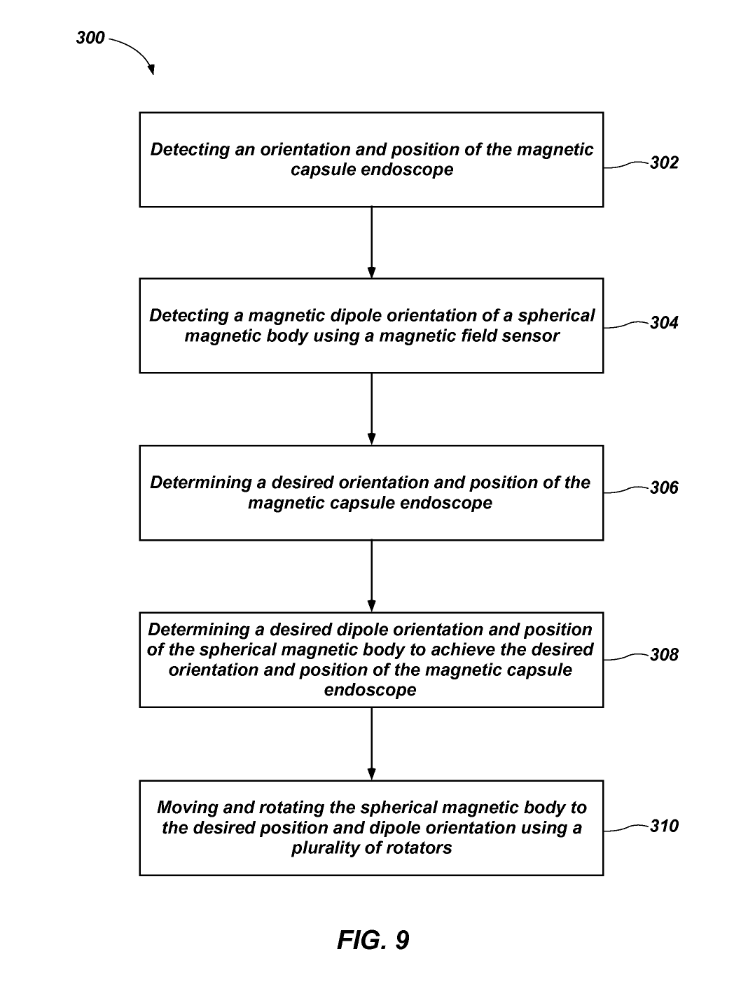

FIG. 9 shows a method 300 in accordance with an embodiment of the present invention. At step 302, the operation is performed of detecting an orientation and position of the magnetic capsule endoscope. At step 304, the operation is performed of detecting a magnetic dipole orientation of a spherical magnetic body using a magnetic field sensor. At step 306, the operation is performed of determining a desired orientation and position of the magnetic capsule endoscope using a processor. At step 308, the operation is performed of determining a desired dipole orientation and position of the spherical magnetic body to achieve the desired orientation and position of the magnetic capsule endoscope using the processor. At step 310, the operation is performed of moving and rotating the spherical magnetic body to the desired position and dipole orientation using a plurality of rotators. These steps can be performed by utilizing all or some of the systems and components discussed in the present invention, with particular reference to FIGS. 1, 2, 5A, and 5B as example embodiments.

While the forgoing examples are illustrative of the principles of the present technology in one or more particular applications, it will be apparent to those of ordinary skill in the art that numerous modifications in form, usage and details of implementation can be made without the exercise of inventive faculty, and without departing from the principles and concepts of the technology. Accordingly, it is not intended that the technology be limited, except as by the claims set forth below.

What is claimed is:

1. A magnetic manipulation device, comprising:
a housing;
a spherical magnetic body contained within the housing, the spherical magnetic body being rotatable about a sphere axis of rotation which is omnidirectionally variable; and
a plurality of rotators in contact with the spherical magnetic body and adapted to rotate the spherical magnetic body about the sphere axis of rotation.

2. The device of claim 1, wherein the plurality of rotators includes three rotators.

3. The device of claim 1, wherein the plurality of rotators are omniwheels.

4. The device of claim 1, wherein the plurality of rotators includes three omniwheels, wherein an axis of rotation of each of the three omniwheels is oriented approximately orthogonal to one another

5. The device of claim 1, further comprising a magnetic field sensor proximal to the spherical magnetic body to measure a dipole orientation of the spherical magnetic body.

6. The device of claim 5, wherein the magnetic field sensor is coupled to the housing.

7. The device of claim 1, wherein the spherical magnetic body comprises a spherical permanent magnet.

8. The device of claim 1, wherein the spherical magnetic body comprises a non-spherical permanent magnet encapsulated in a spherical structure.

9. The device of claim 1, wherein each of the plurality of rotators include a plurality of compressible support arms.

10. The device of claim 9, wherein each of the compressible support arms include a plurality of opposing cutouts to form a serpentine leveraged support arm that is compressible along a support axis extending a length of the support arm.

11. The device of claim 10, further comprising a compliant material substantially filling the cutouts.

12. The device of claim 1, wherein the device is part of a system for manipulation of a magnetic capsule endoscope, the system further comprising a robotic manipulator supporting the housing and a manipulation processor for causing movements of the robotic manipulator and the plurality of rotators.

13. A system for manipulation of a magnetic capsule endoscope, comprising:
a magnetic manipulation device comprising a spherical magnetic body and a plurality of rotators in contact with the spherical magnetic body to rotate the spherical magnetic body in a desired direction;
a robotic arm supporting the magnetic manipulation device, said robotic arm being movable along at least two axes;
a magnetic field sensor proximal to the spherical magnetic body to measure a magnetic dipole direction of the spherical magnetic body; and
a processor for causing movements of the robotic arm and the plurality of rotators based on a location of the magnetic capsule endoscope and the magnetic dipole direction of the spherical magnetic body.

14. The system of claim 13, wherein the magnetic field sensor comprises a plurality of magnetic field sensors.

15. The system of claim 14, wherein the plurality of magnetic field sensors comprises three sensors in close proximity to each other oriented in mutually perpendicular directions.

16. The system of claim 14, wherein the plurality of magnetic field sensors are arranged so that at least one sensor measures a nonzero component of the magnetic field for every possible dipole orientation of the spherical magnetic body.

17. The system of claim 13, wherein the plurality of rotators includes three omniwheels, wherein an axis of rotation of each of the three omniwheels is approximately orthogonal to each of the other two omniwheels.

18. A method of manipulating a magnetic device, comprising:
detecting a magnetic dipole orientation of a spherical magnetic body using a magnetic field sensor;
determining a desired dipole orientation of the spherical magnetic body using a processor; and
rotating the spherical magnetic body within a housing to the desired dipole orientation using a plurality of omniwheels.

19. A method of manipulating a magnetic capsule endoscope, comprising:
detecting an orientation and position of the magnetic capsule endoscope;
detecting a magnetic dipole orientation of a spherical magnetic body using a magnetic field sensor;
determining a desired orientation and position of the magnetic capsule endoscope using a processor;
determining a desired dipole orientation and position of the spherical magnetic body to achieve the desired orientation and position of the magnetic capsule endoscope using the processor; and
moving and rotating the spherical magnetic body to the desired position and dipole orientation using a plurality of rotators.

20. The method of claim 19, wherein a housing encloses the spherical magnetic body, the spherical magnetic body being freely rotatable within the housing, and moving the spherical magnetic body comprises moving a robotic arm supporting the housing.

21. The method of claim 19, wherein detecting the dipole orientation of the spherical magnetic body comprises detecting the dipole orientation using three magnetic field sensors in close proximity to each other oriented in mutually perpendicular directions.

22. The system of claim 19, wherein detecting the dipole orientation of the spherical magnetic body comprises detecting the dipole orientation using a plurality of magnetic field sensors arranged so that at least one sensor measures a nonzero component of the magnetic field for every possible dipole orientation of the spherical magnetic body.
